(19) United States
(12) Patent Application Publication
(10) Pub. No.: US 2011/0276127 A1
(43) Pub. Date: Nov. 10, 2011

(54) MULTIPLE INFLATION OF AN EXPANDABLE MEMBER AS A PRECURSOR TO AN IMPLANT PROCEDURE

(76) Inventors: David C. Forster, Los Altos Hills, CA (US); Yenyu Chen, Palo Alto, CA (US)

(21) Appl. No.: 12/941,854

(22) Filed: Nov. 8, 2010

Related U.S. Application Data
(63) Continuation-in-part of application No. 11/420,189, filed on May 24, 2006.

(51) Int. Cl. A61F 2/24

(52) U.S. Cl. 623/2.11

(57) ABSTRACT

Devices and methods for assessing the orientation and shape of vessel lumens and hollow portions of organs are described. The devices and methods are particularly adapted for determining the orientation and shape of the native heart valves to facilitate the later implantation of a prosthetic heart valve. The devices are typically catheter-based having an expandable member fixed to a distal end of the catheter. The devices and methods typically comprise deploying the expandable member percutaneously to a target location, expanding the expandable member, performing a valvuloplasty procedure to enlarge the diseased lumen and then performing an assessment of the diseased lumen with the expandable member to determine at least one property of the lumen. An implant device is inserted after assessment of the diseased lumen.

### CROSS REFERENCES TO RELATED APPLICATIONS

This application is a continuation-in-part application of U.S. patent application Ser. No. 11/420,189, entitled “Assessment of Aortic Heart Valve to Facilitate Repair or Replacement,” filed May 24, 2006, which application is hereby incorporated by reference in its entirety.

### FIELD OF THE INVENTION

The present invention relates generally to medical devices and methods. More particularly, the present invention relates to methods and devices for assessing the orientation, shape, size, topography, compliance, and other aspects of lumens and surrounding tissue. The devices and methods are particularly adapted for use during minimally invasive surgical interventions, but may also find application during surgical replacement on a stopped heart, less invasive surgical procedures on a beating heart, and other percutaneous procedures.

### BACKGROUND OF THE INVENTION

Minimally invasive surgery provides several advantages over conventional surgical procedures, including reduced recovery time, reduced surgically-induced trauma, and reduced post-surgical pain. Moreover, the expertise of surgeons performing minimally invasive surgery has increased significantly since the introduction of such techniques in the 1980s. As a result, substantial focus has been paid over the past twenty years to devices and methods for facilitating and improving minimally invasive surgical procedures.

One area in which there remains a need for substantial improvement is pre-surgical assessment of treatment locations intended to be subjected to a minimally invasive surgical procedure. For example, when a surgical procedure is to be performed at a treatment location within the body of a patient, it would frequently be beneficial for the surgeon to have prior knowledge of the shape, size, topography, compliance, and other physical properties of the treatment location. This information would be particularly useful in relation to minimally invasive surgical procedures in which prosthetic devices are implanted within a body lumen or within a hollow portion of an organ located within the body of the patient. Such information could then be used to select the size and/or shape of the prosthetic device to more closely match the size, shape, and topography of the treatment location.

A particular portion of the anatomy for which complete and accurate physical assessment would be beneficial are the coronary valves. Diseases and other disorders of heart valves affect the proper flow of blood from the heart. Two categories of heart valve disease are stenosis and incompetence. Stenosis refers to a failure of the valve to open fully, due to stiffened valve tissue. Incompetence refers to valves that cause inefficient blood circulation, permitting backflow of blood in the heart.

Medication may be used to treat some heart valve disorders, but many cases require replacement of the native valve with a prosthetic heart valve. In such cases, a thorough assessment of the shape, size, topography, compliance, and other physical properties of the native valve annulus would be extremely beneficial. Prosthetic heart valves can be used to replace any of the native heart valves (aortic, mitral, tricuspid or pulmonary), although repair or replacement of the aortic or mitral valves is most common because they reside in the left side of the heart where pressures are the greatest.

A conventional heart valve replacement surgery involves accessing the heart in the patent's thoracic cavity through a longitudinal incision in the chest. For example, a median sternotomy requires cutting through the sternum and forcing the two opposing halves of the rib cage to be spread apart, allowing access to the thoracic cavity and heart within. The patient is then placed on cardiopulmonary bypass which involves stopping the heart to permit access to the internal chambers. After the heart has been arrested the aorta is cut open to allow access to the diseased valve for replacement. Such open heart surgery is particularly invasive and involves a lengthy and difficult recovery period.

Less invasive approaches to valve replacement have been proposed. The percutaneous implantation of a prosthetic valve is a preferred procedure because the operation is performed under local anesthesia, does not require cardiopulmonary bypass, and is less traumatic.

### BRIEF SUMMARY OF THE INVENTION

The present invention provides methods and devices for assessing the shape, size, topography, compliance, and other physical properties of a vessel lumen or a hollow portion of another organ located within a patient. The methods and devices may find use in the coronary vasculature, the atrial appendage, the peripheral vasculature, the abdominal vasculature, and in other ducts such as the biliary duct, the fallopian tubes, and similar lumen structures within the body of a patient. The methods and devices may also find use in the heart, lungs, kidneys, or other organs within the body of a patient. Moreover, although particularly adapted for use in vessels and organs found in the human body, the apparatus and methods may also find application in the treatment of animals.

However, the primary use of the methods and devices described herein is in the assessment of the size, shape, topography, compliance, spatial orientation, and other physical properties of the native heart valves. Such assessments are useful to facilitate proper orientation, sizing, selection, and implantation of prosthetic heart valves into the native valve space. Proper orientation, selection and sizing ensures that the prosthetic heart valve that is delivered during the implantation procedure will be of a size and shape that fits within the native valve space, including accommodations for any defects or deformities that are detected by the assessment process. Proper orientation, selection and sizing also ensures that the prosthetic valve, once fully expanded, will properly seal against the aortic wall to prevent leakage, and to prevent migration of the prosthetic valve.

The methods and devices described herein are suitable for use in facilitating the orientation, selection and sizing of prosthetic heart valves of all types, independent of the design, implantation mechanism, deployment technique, or any other aspect of the prosthetic valve. In many cases, particularly in the case of a prosthetic valve that is expandable from a delivery state to a deployed state, the assessment of the native valve space is of very great importance. For example, it is important to know the diameter of the native valve space when the valve space has been placed under the expansive load that is produced by the prosthetic valve. If the valve does not fit properly, it may migrate, leak, or resist deployment altogether.

The methods include use of an assessment member that is preferably located at or near the distal end of a catheter or other similar device. The assessment member is introduced to a treatment location within the patient, preferably the native cardiac valve, where the assessment member is activated or otherwise put into use to perform an assessment of one or more physical parameters of the treatment location, to collect the assessment information, and to provide the assessment information to the clinician. Assessment information includes the size (e.g., diameter, circumference, area, volume, etc.) of the valve space, the shape (e.g., round, spherical, irregular, etc.) of the lumen or hollow portion of the organ, the topography (e.g., locations, sizes, and shapes of any irregular features) of the lumen or hollow portion of the organ, the nature of any regular or irregular features (e.g., thrombosis, calcification, healthy tissue, fibrosa) and the spatial orientation (e.g., absolute location relative to a fixed reference point, or directional orientation) of a point or other portion of the treatment location. Access to the treatment location is obtained by any conventional method, such as by general surgical techniques, less invasive surgical techniques, or percutaneously. A preferred method of accessing the treatment location is transluminally, preferably by well-known techniques for accessing the vasculature from a location such as the femoral artery. The catheter is preferably adapted to engage and track over a guidewire that has been previously inserted and routed to the treatment site.

The assessment mechanism includes an expandable member that is attached to the catheter shaft at or near its distal end. The expandable member may comprise an inflatable balloon, a structure containing a plurality of interconnected metallic or polymeric springs or struts, an expandable “wisk”-like structure, or other suitable expandable member. In the case of an inflatable balloon, the expandable member is operatively connected to a source of inflation medium that is accessible at or near the proximal end of the catheter. The expandable member has at least two states, an unexpanded state and an expanded state. The unexpanded state generally corresponds with delivery of the assessment mechanism through the patient's vasculature. The expanded state generally corresponds with the assessment process. The expandable member is adapted to provide assessment information to the user when the expandable member is engaged with a treatment location within the body of a patient.

Turning to several exemplary devices and methods, in one aspect of the invention, a catheter-based system includes a transluminal imaging device contained partially or entirely within an expandable structure attached at or near the distal end of the catheter.

In the preferred embodiments, the expandable member is a balloon member. The balloon member is connected to an inflation lumen that runs between the proximal and distal ends of the catheter, and that is selectively attached to a source of inflation medium at or near the proximal end of the catheter. The balloon member is thereby selectively expandable while the imaging device is located either partially or entirely within the interior of the balloon. The imaging device is adapted to be advanced, retracted, and rotated within the balloon, thereby providing for imaging in a plurality of planes and providing the ability to produce three-dimensional images of the treatment site.

In optional embodiments, the expandable member is filled with a medium that enhances the imaging process. For example, the medium may comprise a material that increases the transmission capabilities of the ultrasonic waves, or that reduces the amount of scattering of the ultrasonic waves that would otherwise occur without use of the imaging-enhancing medium. In still other optional embodiments, the expandable structure contains (e.g., has embedded or formed within) or is formed of a material that enhances the imaging process. In still other embodiments, the expandable member includes a layer of or is coated with a material that enhances the imaging process.

In use, the transluminal imaging device is first introduced to the target location within the patient, such as the native valve annulus. In the preferred embodiment, this is achieved by introducing the catheter through the patient's vasculature to the target location. Typically, the catheter tracks over a guidewire that has been previously installed in any suitable manner. The imaging device may be provided with a radiopaque or other suitable marker at or near its distal end in order to facilitate delivery of the imaging device to the target location by fluoroscopic visualization or other suitable means. Once the imaging device is properly located at the target location, the expandable structure is expanded by introducing an expansion medium through the catheter lumen. The expandable structure expands such that it engages and applies pressure to the internal walls of the target location, such as the valve annulus. The expandable structure also takes on the shape of the internal surface of the target location, including all contours or other topography. Once the expandable structure has been sufficiently expanded, the imaging device is activated. Where appropriate, the imaging device is advanced, retracted, and/or rotated to provide sufficient movement to allow a suitable image of the target location to be created, or to collect a desired amount of measurement information. The measurement information collected and/or the images created by the imaging device are then transmitted to a suitable user interface, where they are displayed to the clinician.

In use, the expandable member is first introduced to the target location within the patient. In the preferred embodiment, this is achieved by introducing the catheter through the patient's vasculature to the target location. The catheter tracks over a guidewire that has been previously installed in any suitable manner. The expandable member carried on the catheter may be provided with a radiopaque or other suitable marker at or near its distal end in order to facilitate delivery of the physical assessment member to the target location by fluoroscopic visualization or other suitable means. Once the expandable member is properly located at the target location, the expandable member is expanded by introducing an expansion medium through the catheter lumen. The expandable member expands to a predetermined size such that the expandable member is able to engage the lumen or hollow portion of the organ, thereby providing an indicator of the shape and orientation of the lumen or hollow portion of the organ. In this way, the clinician is able to obtain precise measurements of the shape and orientation of the lumen or hollow portion of the organ at the target location. In a further preferred embodiment, the expandable member may be expanded to a size greater than the lumen or hollow portion of the organs to provide additional assessment information.

In a further aspect of the present invention, a valvuloplasty procedure is performed in association with the assessment of a diseased lumen, such as the native cardiac valve. In a first embodiment, the expandable member also functions as a valvuloplasty balloon. The expandable member is placed within the diseased lumen, where it is expanded. Expansion of the expandable member causes the diseased lumen to increase in size and forces the lumen, which is typically in a diseased state in which it is stiff and decreased in diameter, to open more broadly. The valvuloplasty procedure may therefore be performed prior to the deployment of an implant device such as a prosthetic heart valve, but during a single interventional procedure. In an exemplary embodiment, the expandable member that is used for valvuloplasty, or a different expandable member, is expanded in an assessment function to determine at least one property of the diseased lumen. In one preferred embodiment, the at least one property includes determining an expansion of the diseased lumen and the force exerted in the expansion of the diseased lumen and/or the compliance of the diseased lumen. In a further preferred embodiment, the expandable member after performing valvuloplasty may be expanded beyond the shape and size of the diseased lumen to distort the anatomy and perform an assessment function. An implant device may be inserted into the diseased lumen.

The measurement and diagnostic processes performed by any of the foregoing devices and methods may be used to facilitate any suitable medical diagnosis, treatment, or other therapeutic processes. One particular treatment that is facilitated by the foregoing devices and methods is the repair and/or replacement of coronary valves, particularly aortic valve replacement using a prosthetic valve.

Other aspects, features, and functions of the inventions described herein will become apparent by reference to the drawings and the detailed description of the preferred embodiments set forth below.

### DESCRIPTION OF THE DRAWINGS

FIG. 1 is a perspective view of a catheter in accordance with several of the embodiments of the present invention.

FIG. 2A is a cross-sectional view of an imaging device in accordance with the present invention.

FIG. 2B is a cross-sectional view of the imaging device of FIG. 2A, showing an expandable member in its expanded state.

FIG. 3 is a graphical illustration of expansion of a diseased heart valve during valvuloplasty.

FIG. 4 is a graphical illustration of expansion of a diseased heart valve during an assessment subsequent to valvuloplasty.

FIG. 5 is a graphical illustration of multiple expansions of a diseased heart valve and the effect on sizing of a replacement heart valve.

### DETAILED DESCRIPTION OF THE PREFERRED EMBODIMENTS

The present invention is directed to methods and devices for assessing the orientation, shape, size, topography, contours, and other aspects of anatomical vessels and organs using minimally invasive surgical techniques. As summarized above, the devices are typically catheter-based devices. Such devices are suitable for use during less invasive and minimally invasive surgical procedures. However, it should be understood that the devices and methods described herein are also suitable for use during surgical procedures that are more invasive than the preferred minimally invasive techniques described herein.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present inventions.

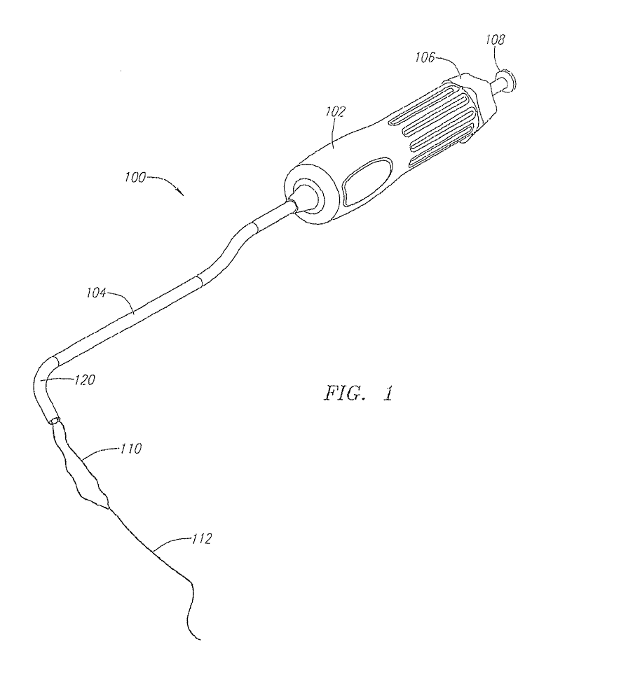

Turning to the drawings, FIG. 1 shows a catheter 100 suitable for use with each of the assessment mechanisms described herein. The catheter 100 includes a handle 102 attached to the proximal end of an elongated catheter shaft 104. The size and shape of the handle 102 may vary, as may the features and functionality provided by the handle 102. In the illustrated embodiment, the handle 102 includes a knob 106 rotatably attached to the proximal end of the handle 102. The knob 106 may be rotated to control the movement and/or function of one or more components associated with the catheter 100, such as for retraction of one or more catheter shafts or sheaths, or manipulation of an expandable member or other component carried at or near the distal end of the catheter shaft 104. Alternative structures may be substituted for the knob 106, such as one or more sliders, ratchet mechanisms, or other suitable control mechanisms known to those skilled in the art.

An inflation port 108 is located near the proximal end of the handle 102. The inflation port 108 is operatively connected to at least one inflation lumen that extends through the catheter shaft 104 to an expandable member 110 located near the distal end of the catheter shaft 104. The inflation port 108 is of any suitable type known to those skilled in the art for engaging an appropriate mechanism for providing an inflation medium to inflate the expandable member 110. For example, a suitable inflation mechanism is an Indeflator™ inflation device, manufactured by Guidant Corporation.

The catheter 100 is adapted to track a guidewire 112 that has been previously implanted into a patient and routed to an appropriate treatment location. A guidewire lumen extends through at least the distal portion of the catheter shaft 104, thereby providing the catheter 100 with the ability to track the guidewire 112 to the treatment location. The catheter 100 may be provided with an over-the-wire construction, in which case the guidewire lumen extends through the entire length of the device. Alternatively, the catheter 100 may be provided with a rapid-exchange feature, in which case the guidewire lumen exits the catheter shaft 104 through an exit port at a point nearer to the distal end of the catheter shaft 104 than the proximal end thereof.

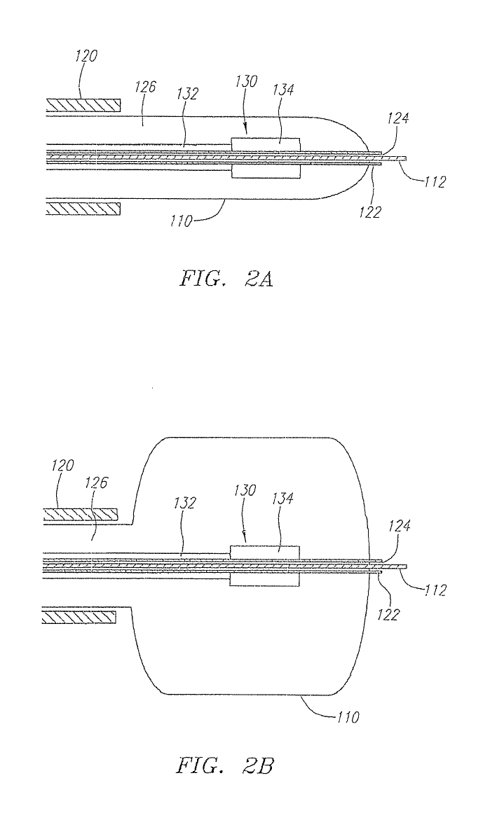

Turning next to FIGS. 2A-B, an assessment mechanism is shown and described. The assessment mechanism is located at the distal end of a catheter 100, such as that illustrated in FIG. 1 and described above. The assessment mechanism shown in FIGS. 2A-B includes an imaging device that is used to provide two-dimensional or three-dimensional images of a vessel lumen or the hollow portion of an organ within the body of a patient, as described below.

The assessment mechanism includes the outer sheath 120 of the catheter shaft 104, which surrounds the expandable member 110. In the preferred embodiment, the expandable member 110 is an inflatable balloon. The expandable member 110 is attached at its distal end to a guidewire shaft 122, which defines a guidewire lumen 124 therethrough. The guidewire 112 extends through the guidewire lumen 124.

An imaging member 130 is contained within the expandable member 110. The imaging member 130 is supported by a shaft 132 that extends proximally to the handle 102, where it is independently controlled by the user. The imaging member shaft 132 is coaxial with and surrounds the guidewire shaft 124, but is preferably movable (e.g., by sliding) independently of the guidewire shaft 124. At the distal end of the imaging member shaft 132 is the imaging head 134. The imaging head 134 may be any mechanism suitable for transmitting and receiving ultrasonic waves. A typical imaging head 134 is an ultrasonic imaging probe for ultrasound imaging. It is within the scope of the present invention to have other imaging members 130. Such other imaging members 130 may include, but are not limited to, an optical fiber in conjunction with optical coherence tomography for optical imaging or an acoustic imaging device for tranesophageal echo. The expandable member 110 is subject to expansion when a suitable expansion medium is injected into the expandable member through the inflation lumen 126. The inflation lumen 126, in turn, is connected to the inflation port 108 associated with the handle 102. FIG. 2A illustrates the expandable member 110 in its unexpanded (contracted) state, while FIG. 2B illustrates the expandable member 110 in its expanded state, such as after a suitable inflation medium is injected through the inflation port 108 and inflation lumen 126 into the expandable member 110.

To use the assessment mechanism illustrated in FIGS. 2A-B, the distal portion of the catheter is delivered to a treatment location within the body of a patient over the previously deployed guidewire 112. In a particularly preferred embodiment, the treatment location is the aortic heart valve, and the guidewire 112 is deployed through the patient's vasculature from an entry point in the femoral artery using, for example, the Seldinger technique. Deployment of the assessment mechanism is preferably monitored using fluoroscopy or other suitable visualization mechanism. Upon encountering the treatment location, the expandable member 110 is expanded by inflating the balloon with a suitable inflation medium through the inflation port 108 and the inflation lumen 126. The expandable member 110 engages the internal surfaces of the treatment location, such as the annular root of the aortic heart valve. Once the expandable member 110 is expanded, the imaging head 134 is activated and the imaging process is initiated. The imaging head 134 is preferably advanced, retracted, and rotated within the expandable member 110 as needed to obtain images in a variety of planes to yield a 360° three-dimensional image, or any desired portion thereof. Once the imaging process is completed, the expandable member 110 is deflated, and the assessment mechanism may be retracted within the catheter shaft 104. The catheter 100 is then removed from the patient.

Optionally, the inflation medium used to expand the expandable member 110 may comprise a material that enhances the ability of the imaging head 134 to generate images. For example, the inflation medium may facilitate enhanced acoustic transmission, reception, or it may reduce the incidence of scattering of the assessment signal. Such suitable inflation media may include a liquid or a gas and, more specifically, may include, for example, the following: acoustic gel, dielectric fluid, saline, blood, gas, contrast medium and the like. These effects may be enhanced further by provision of a material or coating on the surface of the expandable member 110 that optimizes the imaging process. Such suitable materials and/or coatings include relatively dense materials such as metal, ceramic, high density polymers, and the like.

Heart valves are placed within the human body to replace a diseased heart valve in a two step procedure. The first step is a valvuloplasty procedure to open the diseased heart valve and the second step is inserting the heart valve in the opening formed by the valvuloplasty procedure. The valvuloplasty procedure opens the diseased heart valve by “pushing” the plaque (i.e., calcification) out of the diseased heart valve and into the surrounding tissue. Similar medical treatment is done in the case of less invasive valve replacement, stents and other medical procedures.

It is becoming increasingly important to understand the relationship, interaction and function between a medical implant or other device and the physical anatomy with which it interfaces. An implant must be properly sized with respect to the size of the anatomy so that there is no leakage, movement or damage to the surrounding tissue. In the case of a heart valve, very diseased tissue can be so stiff that it can distort or collapse the deployed diameter of the replacement heart valve to a smaller or distorted condition. Distortion and/or reduction in deployment diameter is known to adversely affect tissue leaflet durability and life expectancy of the replacement heart valve.

In the present exemplary embodiments, an expandable member is inflated and caused to modify the human anatomy prior to placement of an implant device such as a heart valve. After modification of the human anatomy, a subsequent assessment step will be performed to determine one or more properties of the human anatomy prior to placement of the implant device. These properties may include the force to expand the expandable member, the size of the human anatomy and the compliance of the human anatomy. The expandable member may be deflated prior to the subsequent assessment step to allow for blood to flow. It is believed that the properties of the human anatomy will be affected by the modification of the human anatomy so that knowing the post-modification anatomy physical properties is important as it is in this state that any implant device would actually be placed. It is also desirable to understand the human anatomy prior to and after modification to determine if there is any correlation between the pre-modification and post-modification states of the human anatomy.

In an exemplary embodiment, a valvuloplasty procedure is performed wherein the expandable member is placed within the cardiac valve space, where it is expanded. Expansion of the expandable member causes the native valve to increase in size and forces the valve, which is typically in a diseased state in which it is stiff and, decreased in diameter, to open more broadly.

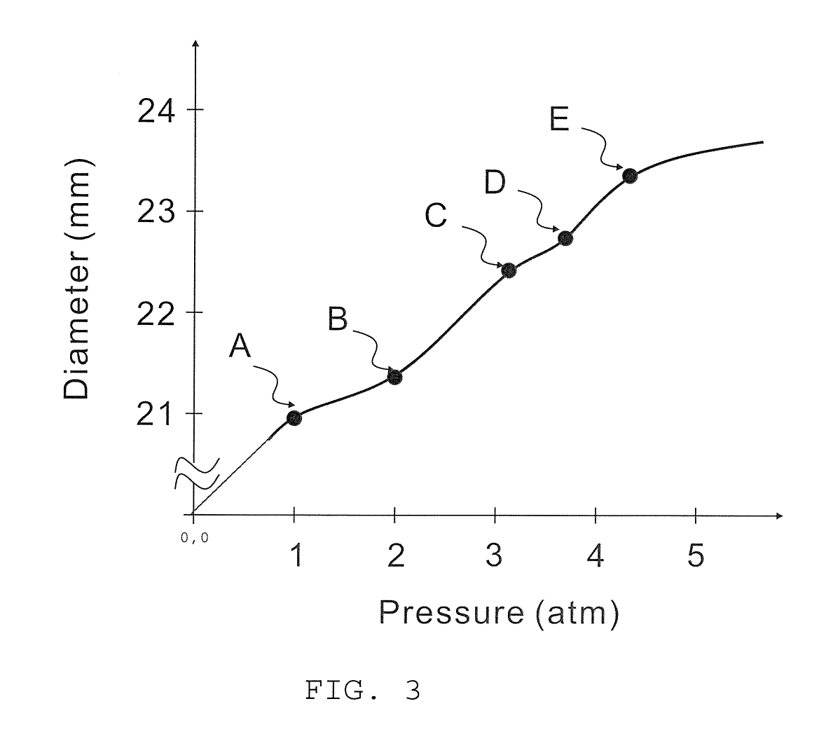

The following discussions regarding FIGS. 3, 4, and 5 are prophetic examples.

FIG. 3 is a graphical illustration of a valvuloplasty procedure for a diseased heart valve. Starting at point 0,0 in FIG. 3, there is no pressure and little volume in the expandable member 110. The expandable member 110 expands until the expandable member 110 contacts the aortic wall at point A. In the interval from point 0,0 to point A, there is little increase in pressure as the expandable member 110 expands without resistance and the line from point 0,0 to point A is steady.

When the expandable member 110 comes in contact with the aortic wall, the expandable member 110 begins to push the material making up the aortic root/annulus, thus performing valvuloplasty. Increased volume and pressure are required to achieve an effective clinical result. Referring again to FIG. 3, calcium fracturing may occur at point B and again at points C and D.

At point D shown in FIG. 3, all modes of valvuloplasty may have been completed.

At point E in FIG. 3, there is rapid increase in pressure with little increase in volume which validates that valvuloplasty is completed and further expansion of the expandable member 110 could cause tearing of the aortic wall. The expanding of the expandable member 110 is then halted and then the deflation and subsequent withdrawal of the expandable member 110 occurs.

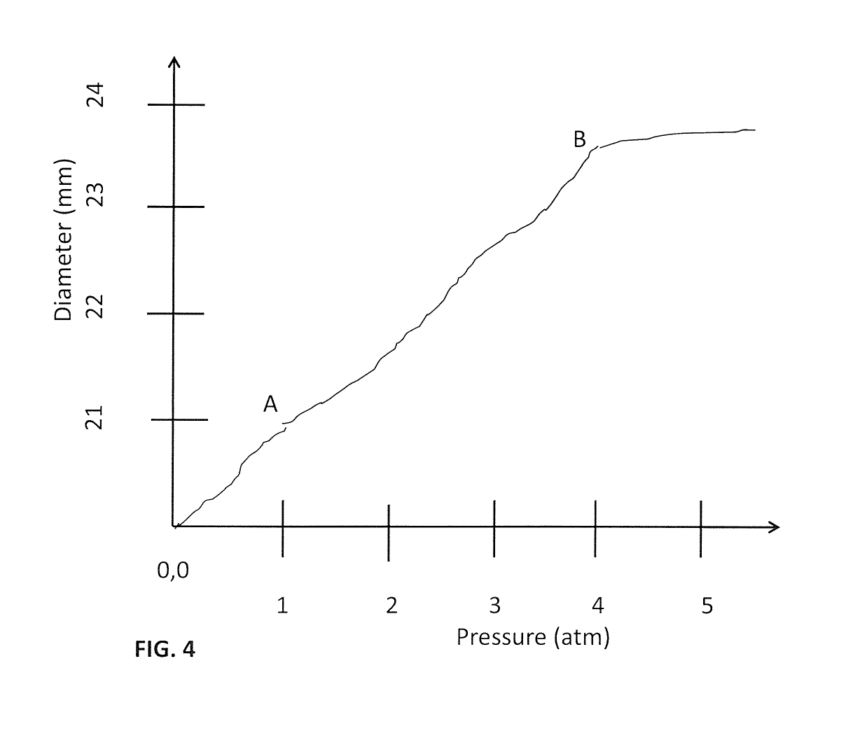

FIG. 4 is a graphical illustration of an assessment step after the valvuloplasty step described with respect to FIG. 3. If the human anatomy were to be reassessed after the initial valvuloplasty, it is believed that the human anatomy would respond differently in the reassessment procedure than in the valvuloplasty due to most, if not all, of the calcification being cracked during the valvuloplasty. Starting at point 0,0 in FIG. 4, there is no pressure and little volume in the expandable member 110. The expandable member 110 expands until the expandable member 110 contacts the aortic wall at point A. In the interval from point 0,0 to point A, there is little increase in pressure as the expandable member 110 expands without resistance and the line from point 0,0 to point A is steady.

Similarly when the expandable member 110 comes in contact with the aortic wall at point A, the expandable member 110 expands with a proportionate increase in pressure so that the line from point A to point B is steady. At point B in FIG. 4, there is rapid increase in pressure with little increase in volume which indicates that further expansion of the expandable member 110 could cause tearing of the aortic wall. The expanding of the expandable member 110 is then halted and then the deflation and subsequent withdrawal of the expandable member 110 occurs.

Comparing FIGS. 3 and 4, it can be seen that it is believed that the maximum diameter at point E in FIG. 3 is reached at about 4.5 atmospheres rather than 4 atmospheres at point B in FIG. 4, thereby indicating that the human anatomy has changed after the valvuloplasty procedure in FIG. 3.

While the above prophetic examples have been illustrated using one valvuloplasty procedure and one assessment procedure, it can be envisioned that in some cases it may be necessary to perform more than one valvuloplasty procedure followed by the assessment procedure to stabilize the human anatomy.

It can be appreciated that if an implant device were to be implanted based on the results in FIG. 3 instead of FIG. 4, there could very well be improper fit of the implant device in the patient. Accordingly, the assessment step is desirable to obtain the current state of the human anatomy before the implant device is implanted. Knowing the post-valvuloplasty anatomy properties allows one to make size, type and manufacturer selections, predict safety and efficacy and also to correctly insert the implant device.

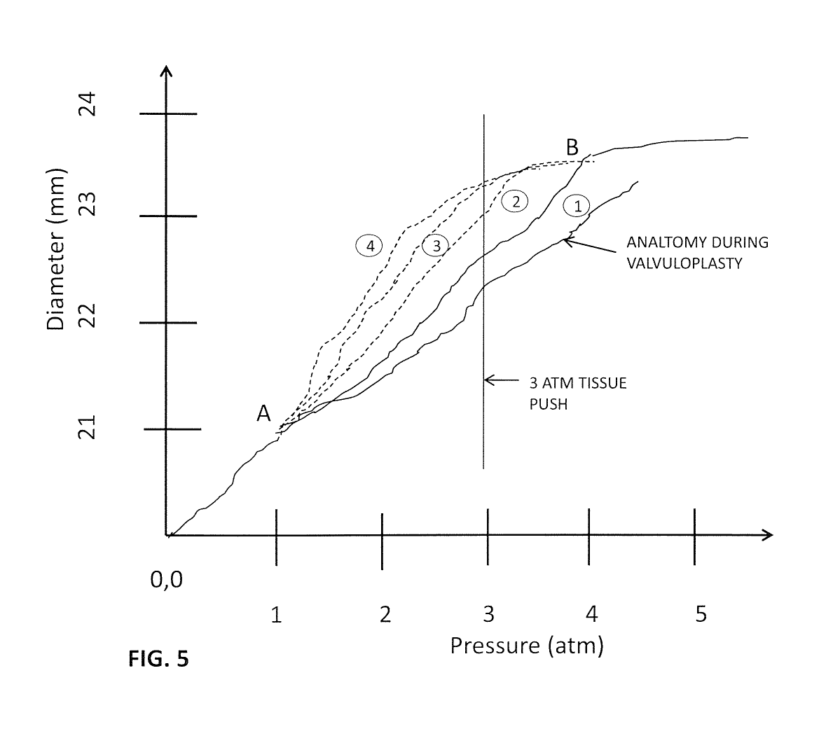

Referring now to FIG. 5, the graphs of FIGS. 3 and 4 have been overlaid and additional valvuloplasty procedures have been performed. It can be seen that with multiple valvuloplasty procedures, the slope of the curves from A to B becomes steeper and the curves move closer together with each valvuloplasty procedure. After some number of valvuloplasty procedures, the curves would not change with further valvuloplasty procedures. The importance of multiple valvuloplastys becomes apparent when sizing for the replacement heart valve. If a tissue push to 3 atmospheres is assumed, a heart valve having a diameter of about 22.25 mm would be needed after the first valvuloplasty. This, however, may not be the best fit for the heart valve. After a second valvuloplasty (curve 1), a heart valve of about 22.5 mm may be needed. After further valvuloplastys (curves 2, 3, and 4), a heart valve of about 23.25 mm may be needed. Accordingly, multiple valvulplasty procedures affect the choice of heart valve size.

The preferred embodiments of the inventions that are the subject of this application are described above in detail for the purpose of setting forth a complete disclosure and for the sake of explanation and clarity. Those skilled in the art will envision other modifications within the scope and spirit of the present disclosure. Such alternatives, additions, modifications, and improvements may be made without departing from the scope of the present inventions, which is defined by the claims.

What is claimed is:

1. A method of inserting an implant device comprising:
performing a first valvuloplasty with a first expandable member to enlarge a diseased lumen in a human patient;
subsequent to the first valvuloplasty, performing an assessment of the diseased lumen with a second expandable member to determine at least one property of the diseased lumen;
choosing an implant device based on the at least one property; and
inserting the implant device.

2. The method of claim 1 further comprising a medium for expanding the first expandable member.

3. The method of claim 2 wherein the medium is a liquid or a gas.

4. The method of claim 2 wherein the medium is selected from the group consisting of saline, acoustic gel, dielectric fluid, blood, gas and contrast medium.

5. The method of claim 1 wherein the first and second expandable members comprise a balloon.

6. The method of claim 1 wherein the first and second expandable members are the same expandable member.

7. The method of claim 1 wherein between the steps of choosing and inserting, further comprising the step of orienting the device based on the at least one property.

8. The method of claim 1 wherein the step of choosing includes choosing a size, type or manufacturer of the implant device.

9. The method of claim 1 wherein the at least one property includes a force exerted in the second expandable member to enlarge the lumen, a size of the diseased lumen or a compliance of the diseased lumen.

10. The method of claim 1 further comprising determining if a correlation exists between the first valvuloplasty and the performing an assessment of the diseased lumen.

11. The method of claim 1 wherein after the step of performing the first valvuloplasty, further comprising deflating the first expandable member prior to performing an assessment of the diseased lumen.

12. The method of claim 1 wherein prior to choosing a size or type of implant device,
performing a second valvuloplasty with the first expandable member to enlarge a diseased lumen; and
subsequent to the second valvuloplasty, performing an assessment of the diseased lumen with the second expandable member to determine at least one property of the diseased lumen.

13. The method of claim 1 wherein prior to choosing a size or type of implant device,
performing a further valvuloplasty with the first expandable member to enlarge the diseased lumen; and
subsequent to the further valvuloplasty, performing an assessment of the diseased lumen with the second expandable member to determine at least one property of the lumen.
repeating performing a further valvuloplasty and performing an assessment of the diseased lumen until the at least one property is within acceptable limits.

14. The method of claim 1 wherein the assessment includes expanding the second expandable member a first time to determine an expansion of the lumen and the force exerted in the expansion of the lumen.

15. The method of claim 1 wherein the first and second expandable members are the same and wherein the assessment includes expanding the second expandable member a second time to determine an expansion of the lumen and the force exerted in the expansion of the lumen.

16. A method of repairing a diseased lumen comprising:
deploying a first expandable member percutaneously to a target location in a human patient,
expanding the first expandable member until the expandable member contacts a wall of a diseased lumen;
performing a first valvuloplasty with the first expandable member to enlarge a diseased lumen;
subsequent to the first valvuloplasty, deploying a second expandable member percutaneously to the target location and performing an assessment of the diseased lumen with the second expandable member to determine at least one property of the lumen;
choosing an implant device based on the at least one property; and
inserting the implant device.

17. The method of claim 16 further comprising a medium for expanding the first expandable member.

18. The method of claim 17 wherein the medium is a liquid or a gas.

19. The method of claim 17 wherein the material is selected from the group consisting of saline, acoustic gel, dielectric fluid, blood, gas and contrast medium.

20. The method of claim 16 wherein the first and second expandable members comprise a balloon.

21. The method of claim 16 wherein the first and second expandable members are the same expandable member.

22. The method of claim 16 wherein between the steps of choosing and inserting, further comprising the step of orienting the device based on the at least one property.

23. The method of claim 16 wherein the step of choosing includes choosing a size, type or manufacturer of the implant device.

24. The method of claim 16 wherein the at least one property includes a force exerted in the second expandable member to enlarge the lumen, a size of the diseased lumen or a compliance of the diseased lumen.

25. The method of claim 16 further comprising determining if a correlation exists between the first valvuloplasty and the performing an assessment of the diseased lumen.

26. The method of claim 16 wherein after the step of performing the first valvuloplasty, further comprising deflating the first expandable member prior to performing an assessment of the diseased lumen.

27. The method of claim 16 wherein prior to choosing a size or type of implant device,
performing a second valvuloplasty with the expandable member to enlarge a diseased lumen; and
subsequent to the second valvuloplasty, performing an assessment of the diseased lumen with the second expandable member to determine at least one property of the lumen.

28. The method of claim 16 wherein prior to choosing a size or type of implant device,
performing a further valvuloplasty with the first expandable member to enlarge the diseased lumen; and
subsequent to the further valvuloplasty, performing an assessment of the diseased lumen with the second expandable member to determine at least one property of the lumen;
repeating performing a further valvuloplasty and performing an assessment of the diseased lumen until the at least one property is within acceptable limits.

29. The method of claim 16 wherein the assessment includes expanding the second expandable member a first time to determine an expansion of the lumen and the force exerted in the expansion of the lumen.

30. The method of claim 16 wherein the first and second expandable members are the same and wherein the assessment includes expanding the second expandable member a second time to determine an expansion of the lumen and the force exerted in the expansion of the lumen.
